(19) United States
(12) Patent Application Publication
(10) Pub. No.: US 2017/0298406 A1
(43) Pub. Date: Oct. 19, 2017

(54) SALT OF PHENYLGLYCINE METHYL ESTER

(71) Applicant: DSM Sinochem Pharmaceuticals Netherlands B.V., DELFT (NL)

(72) Inventor: Thomas Van Der Does, Echt (NL)

(21) Appl. No.: 15/513,156

(22) PCT Filed: Sep. 17, 2015
(86) PCT No.: PCT/EP2015/071324

§ 371 Date: Mar. 21, 2017

(30) Foreign Application Priority Data
Sep. 22, 2014 (EP) 14185735.9

(51) Int. Cl. C12P 35/04; C07C 227/42; C07C 229/36; C07C 227/18

(52) U.S. Cl. CPC C12P 35/04; C12Y 305/01011; C07C 227/18; C07C 227/42; C07C 229/36

(57) ABSTRACT

The present invention relates to the hemi sulfuric acid salt of D-phenylglycine methyl ester, to a method for the preparation of said salt and to the use of said salt in the enzymatic synthesis of antibiotics and of D-phenylglycine methyl ester free base.

### FIELD OF THE INVENTION

The present invention relates to the hemi sulfuric acid salt of D-phenylglycine methyl ester, to a method for the preparation of said salt and to the use of said salt in the enzymatic synthesis of antibiotics.

### BACKGROUND OF THE INVENTION

Enzymatic production of semisynthetic β-lactam antibiotics by acylation of the parent amino β-lactam moiety with a side chain acid derivative, such as an amide or an ester, has been widely described in the patent literature e.g. DE 2163792, DE 2621618, EP 339751, EP 473008, U.S. Pat. No. 3,816,253, WO 92/01061, WO 93/12250, WO 96/02663, WO 96/05318, WO 96/23796, WO 97/04086, WO 98/56946, WO 99/20786, WO 2005/00367, WO 2006/069984 and WO 2008/110527. The enzymes used in the art are in most cases penicillin acylases obtained from Escherichia coli and are immobilized on various types of water-insoluble materials (e.g. WO 97/04086).

Due to the sensitive nature of biocatalysts, enzymatic processes usually have strict requirements with regard to the presence of contaminants. Often, unwanted impurities disturb the proper functioning of an enzyme. For this reason, also in the enzymatic production of semisynthetic β-lactam antibiotics by acylation of the parent amino β-lactam moiety with a side chain acid derivative, such as an amide or an ester, the starting materials are preferably in the highest possible purity. The latter is usually achieved by isolating the starting materials, preferably by means of crystallization. For example, for D-4-hydroxyphenylglycine, the side chain for antibiotics such as amoxicillin, cefadroxil and cefprozil, crystallization of activated derivatives such as amides or esters can be easily achieved. For D-phenylglycine, the side chain for antibiotics such as ampicillin, cefaclor and cephalexin, this is however a major problem. Up to now there have not been any reports on the isolation of crystalline D-phenylglycine methyl ester, one of the most favored starting materials in enzymatic production of ampicillin, cefaclor and cephalexin. As described in WO 2008/110527, there is however a need for highly purified D-phenylglycine methyl ester, as the presence of traces of D-phenylglycine has a strong negative effect on the yield of the enzymatic coupling reaction. This is attributed to the fact that, due to the low solubility of the free side chains under the conditions of the enzymatic coupling reaction, there is an upper limit to the concentration of free side chain in the enzymatic coupling reaction. This limit is determined by the requirement that the free side chain should not crystallize or precipitate, because the precipitate negatively affects the processing of the enzymatic coupling reaction. Moreover, in the final steps of the downstream processing of the semi synthetic β-lactam compound, the contaminating D-phenylglycine has to be removed, for instance with the mother liquor of a final crystallization step of the semi synthetic β-lactam compound. At higher levels of D-phenylglycine, more mother liquor is required to remove the D-phenylglycine which in turn is responsible for higher losses of the semi synthetic β-lactam compound. The unit operation which results in the isolation of the side chain ester in solid form complicates the production process of the semi synthetic antibiotic and significantly contributes to the cost price thereof. Therefore the amount of unwanted D-phenylglycine in D-phenylglycine methyl ester should be as low as possible.

In order to achieve this, D-phenylglycine methyl ester can be isolated in the form of a salt. Several salts such as alkyl- or aryl sulfonic acid salts and the hydrochloric acid have been reported and through such isolation process unwanted traces of D-phenylglycine can be removed. However, these salts bring certain disadvantages such as the introduction of new organic impurities salt. In principle the hydrochloric acid salt is an attractive candidate for isolation of a purified derivative of D-phenylglycine methyl ester but unfortunately, the penicillin acylases are a class of enzymes that is negatively influenced by the presence of chloride salts and therefore the use of the hydrochloric acid salt of D-phenylglycine methyl ester in enzymatic synthesis is accompanied with additional problems that are of a larger magnitude than the problem originally set out to solve. It is for this reason that there remains a need for derivatives of D-phenylglycine methyl ester that can be isolated, are of sufficient purity and do not have the problem associated with the hydrochloric acid salt of D-phenylglycine methyl ester.

### DETAILED DESCRIPTION OF THE INVENTION

It is an object of the present invention to provide a derivative of D-phenylglycine methyl ester that can be isolated, is of sufficient purity and can be used without inhibiting side effects in enzymatic processes leading to ampicillin, cefaclor and cephalexin.

The term “nucleus” is defined herein as the β-lactam moiety of the semi synthetic β-lactam and may be any penem or cephem, for instance 6-aminopenicillanic acid (6-APA), 7-aminodeacetoxy-cephalosporanic acid (7-ADCA), 7-aminocephalosporanic acid (7-ACA) or 7-amino-3-chloro-3-cephem-4-carboxylate (7-ACCA).

The term “side chain” is defined herein as the moiety which in the semi synthetic β-lactam compound is attached to the 6-amino or 7-amino position in the nucleus as defined herein, i.e. D-phenylglycine in ampicillin, cefaclor and cephalexin.

The term “free side chain” is the un-derivatized form of the side chain, i.e. D-phenylglycine.

The term “side chain ester” is the ester form of the free side chain whereby the carboxyl group of the free side chain is esterified to an alcohol, for instance D-phenylglycine methyl ester. The side chain ester may be in the form of the free base or as a salt, for instance as the sulfuric acid salt.

The term “hemi sulfuric acid salt of D-phenylglycine methyl ester”, abbreviated as (PGMH)₂SO₄, refers to the compound of formula (1), with formula C₁₈H₂₄N₂SO₈.

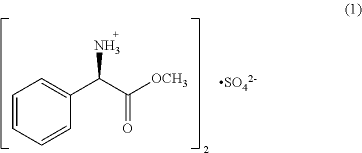

In a first aspect, the invention provides the hemi sulfuric acid salt of D-phenylglycine methyl ester ((PGMH)₂SO₄) in isolated form. Preferably said (PGMH)₂SO₄ is crystalline. In one embodiment crystalline (PGMH)₂SO₄ has an XRD powder diffraction pattern as given in FIG. 1. Preferably said XRD powder diffraction pattern reveals peaks at 6.1±0.2 degrees 2-theta, 12.1±0.2 degrees 2-theta, 18.8±0.2 degrees 2-theta and 24.1±0.2 degrees 2-theta. More preferably said XRD powder diffraction pattern reveals additional peaks at 7.9±0.2 degrees 2-theta, 14.4±0.2 degrees 2-theta, 15.6±0.2 degrees 2-theta, 16.7±0.2 degrees 2-theta, 19.5±0.2 degrees 2-theta and 25.6±0.2 degrees 2-theta.

The (PGMH)₂SO₄ of the present invention advantageously is a stable solid. The only other known stable inorganic acid salt of D-phenylglycine methyl ester is the hydrochloric acid salt. However the latter salt has some drawbacks such as a negative influence on enzyme performance and release of corrosive chloride as side product. The formation of chlorides is known to have a detrimental effect on industrial reactors and this phenomenon does not occur with the sulfates that are being formed with the use of the (PGMH)₂SO₄ of the present invention. Surprisingly, application of the (PGMH)₂SO₄ of the present invention in the enzymatic synthesis of semi synthetic D-phenylglycine-comprising β-lactam compounds such as ampicillin, cefaclor or cephalexin resulted in superior results when compared to the use of a solution of the sulfuric acid salt of D-phenylglycine methyl ester as advocated in U.S. Pat. No. 8,541,199. In one embodiment, the antibiotic cephalexin can be prepared enzymatically from 7-ADCA in higher yields, with higher conversion and lower formation of unwanted D-phenylglycine using the (PGMH)₂SO₄ of the present invention.

In a second aspect, the invention provides a method for the preparation of (PGMH)₂SO₄ comprising the steps of:
(a) contacting a solution of D-phenylglycine methyl ester in an organic solvent with sulfuric acid;
(b) isolating the hemi sulfuric acid salt of D-phenylglycine methyl ester from the mixture obtained in step (a).

Preferably, the amount of sulfuric acid is chosen such that the molar amount of sulfuric acid is from 0.4 to 0.6 relative to the molar amount of (PGMH)₂SO₄. In a preferred embodiment, (PGMH)₂SO₄ is isolated by separating the aqueous phase in step (a) and crystallizing (PGMH)₂SO₄ therefrom. Crystallization may be carried out according to methods known to the skilled person, for example by lowering the temperature. It was found that a preferred crystallization temperature is from −5 to 15° C., more preferably from 0 to 10° C.

In one embodiment, it was found that the overall yield can be improved by recycling the aqueous phase remaining after the isolation in step (b) of the above method. Thus, the aqueous mother liquor is added to the mixture of step (a) in a next cycle of the method as described above. Preferably recycling is carried out such that part of the aqueous mother liquor is discarded prior to addition to the mixture of step (a). A suitable small part is from 1 to 50% by volume, preferably from 2 to 25% by volume, more preferably from 3 to 15% by volume As a result of the phase separation it was found that this recycling can be performed without accumulation of impurities.

The method of the second aspect can be carried out with various organic solvents. It was found that preferred solvents are those having a solubility in water of from 0% (w/w) to 25% (w/w) and having a polarity index of from 1 to 5. Preferably said polarity index is from 2 to 3 as this generally leads to the best results. Preferred solvents are butyl acetate, diethyl ether, ethyl acetate, methyl isobutyl ketone and methyl tert-butyl ether.

In a third aspect, the invention provides the use of (PGMH)₂SO₄ in the preparation of ampicillin, cefaclor or cephalexin comprising contacting said (PGMH)₂SO₄ with 6-aminopenicillanic acid (6-APA), 7-amino-3-chloro-3-cephem-4-carboxylate (7-ACCA) or 7-aminodeacetoxycephalosporanic acid (7-ADCA), respectively in the presence of a penicillin acylase, preferably an immobilized penicillin acylase. This enzymatic reaction may be carried according to any of the processes known in the art and which have been cited hereinbefore. For instance, the synthesis of ampicillin may be carried out as described in EP 339751 or WO 98/56946. Likewise, the synthesis of cephalexin may be carried out as described in WO 96/23796. The synthesis of cefaclor may be carried out as has been described in WO 2006/069984.

After the enzymatic coupling, the semi synthetic beta-lactam antibiotic can be recovered using known methods. For instance, the enzyme reactor may be discharged through the bottom sieve using upwards stirring. The resulting semi synthetic beta-lactam antibiotic suspension may then be filtered through a glass filter.

Due to the low amount of free side chain present after the enzymatic coupling reaction, crystallization of the final semi synthetic beta-lactam antibiotic may be carried out at high concentrations of the beta-lactam antibiotic which results in high yields.

In another embodiment, the third aspect of the invention provides the use of the hemi sulfuric acid salt of D-phenylglycine methyl ester in the preparation of D-phenylglycine methyl ester free base. Such use can be achieved successfully according to the procedure as outlined in WO 2008/110527 for the methyl sulfate of D-phenylglycine methyl ester. It was found that use of the hemi sulfuric acid salt of D-phenylglycine methyl ester of the present invention gives superior results in this respect as compared to the preparation of D-phenylglycine methyl ester free base as described in WO 2008/110527 due to a decrease in mother liquor losses of d-phenylglycine methyl ester free base

### LEGEND TO THE FIGURES

FIG. 1 is the XRD spectrum of the hemi sulfuric acid salt of D-phenylglycine methyl ester. X-axis: 2-theta value (deg). Y-axis: intensity (cps). The following distinct peaks can be discerned:

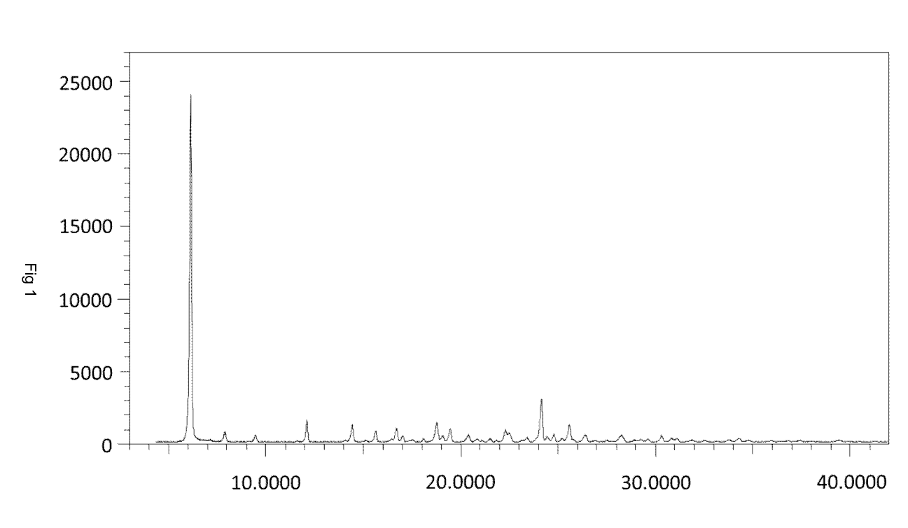

| Peak no. | 2-Theta (deg) | Flex width | d-Value | Intensity | I/Io |
|---|---|---|---|---|---|
| 1 | 6.102 | 0.107 | 144.744 | 24164 | 100 |
| 2 | 7.866 | 0.128 | 112.307 | 739 | 3 |
| 3 | 12.081 | 0.104 | 73.199 | 1445 | 6 |
| 4 | 14.428 | 0.122 | 61.340 | 1251 | 5 |
| 5 | 15.623 | 0.136 | 56.677 | 762 | 3 |
| 6 | 16.683 | 0.134 | 53.098 | 972 | 4 |
| 7 | 18.772 | 0.158 | 47.234 | 1367 | 6 |
| 8 | 19.459 | 0.131 | 45.580 | 967 | 4 |
| 9 | 24.138 | 0.138 | 36.841 | 2997 | 12 |
| 10 | 25.577 | 0.163 | 34.791 | 1219 | 5 |

### EXAMPLES

### General

### X-Ray Powder Diffraction Analysis

A sample was loaded onto a closed sample holder with inner knife (to minimize background scattering) and cavity (diameter 2 cm). The loading was carried out in a fume hood without grinding, in order to minimize dust formation during the sample preparation. Samples were analyzed on an X-ray powder diffractometer D2 Phaser from Bruker. It uses a LynxEye detector with 1° opening angle, a 0.1 mm receiving slit and a nickel filter. The diffraction angle 2∂ ranges from 2° to 60°, with step (in 2θ)˜0.008° and the count time 4 s/step. The sample rotates at 15 rpm during the measurement (for good statistics) and the data are approximately background subtracted.

### HPLC Analysis

Column: HPLC column Crownpak CR(-) (DAICEL), length 150 mm, diameter 4 mm, diameter of particles 5 μm.
Eluent: Solution of HClO₄, pH=2.0. Weigh 1.43 g HClO4 (70%, 1.43 g) was diluted with water for chromatography to 1000 ml and the pH of the solution was checked.
   Chromatographic conditions:
   Eluent: HClO₄, pH=2
   Isocratic conditions
   Flow: 1.0 ml·min⁻¹
   Injection volume: 20 μl
   Wavelength: 220 nm
   Temperature of column: room temperature, 20-25° C.
   Time of chromatogram: 30 min
   Retention times (approximately):
      L-phenylglycine: 2.7 min
      D-phenylglycine: 8.7 min
      L-phenylglycine methyl ester: 9.3 min
      D-phenylglycine methyl ester: 21.0 min

### Preparation of an Aqueous Solution of D-Phenylglycine Methyl Ester (See Also WO 2008/110527, the Similar Procedure of Example 8 of U.S. Pat. No. 8,541,199, with Different Amounts Also Leads to the Same Product and was Used for Example 4)

D-phenylglycine (PG; 135 g) was suspended in methanol (252 mL) and concentrated sulfuric acid (98%, 107 g) was added. The mixture was kept at reflux for 2 hours at approximately 73° C. and concentrated at a reduced pressure using a vacuum pump. The pressure dropped from atmospheric to 20 mbar while at the same time the temperature of the reaction mixture increased from 40 to 80° C. Methanol (126 mL, 100 g) was added and the mixture was kept at reflux for 1 hour at approximately 81° C. and concentrated as described before. The procedure was repeated for another four times (addition of methanol, reflux and concentrating). Finally, methanol (126 mL) was added and the solution was refluxed for another hour and cooled to ambient temperature. Ammonia (15 mL) was dosed with constant rate in 35 min up to pH 2.3-2.4. Water (75 mL) was added and methanol was distilled off at reduced pressure and a temperature below 50° C. The pH of the final D-phenylglycine methyl ester (PGM) solution was 2.0 and the conversion was 99.0%.

### Example 1

### Preparation of Seed of (PGMH)₂SO₄

An aqueous solution of D-phenylglycine methyl ester, obtained as described in the General section (1800 g) was added to a mixture of methyl tert-butyl ether (900 ml) and water (25 ml) at 5-10° C. while the pH was maintained at 9.2 by addition of 8 M NaOH. The phases were separated. The aqueous phase was extracted with methyl tert-butyl ether (600 ml). Both organic phases were combined and added to water (5 mL) while maintaining the pH at 4.2 by addition of 48% (w/w) H₂SO₄. The phases were separated. A viscous, oily water phase (turbid) was obtained. Part of the mixture was evaporated under vacuum (2 mbar) at 20° C. until the weight did not decrease anymore. A viscous oil was obtained. Upon storage at 20°, in the course of days, crystals formed in the oil. Some of these crystals were used to seed the rest of the aqueous phase (in the meantime stored at 3° C.). Very slow crystallization at 3° C. was observed. The crystal suspension was filtered. The crystals were analyzed with HPLC. It turned out that the crystals were contaminated with D-phenylglycine. In the filtrate, crystals formed again upon standing overnight at room temperature. These crystals were isolated, and used as seed in subsequent experiments.

### Example 2

### Preparation of (PGMH)₂SO₄

An aqueous solution of D-phenylglycine methyl ester, obtained as described in the General section (1800 g) was added to a mixture of methyl tert-butyl ether (900 ml) and water (25 ml) at 5-10° C. while the pH was maintained at 9.2 by addition of 8 M NaOH. The phases were separated. The aqueous phase was extracted with methyl tert-butyl ether (600 ml). Both organic phases were combined. The organic phase was determined by HPLC to contain 350.4 g of D-phenylglycine methyl ester. The organic phase was added to water (5 mL) while maintaining the pH at 4.2 by addition of 48% (w/w) H₂SO₄. The consumption of 48% (w/w) H₂SO₄ was 201.7 g. The molar ratio of D-phenylglycine methyl ester (350.4 g, 2.1 mol) and H₂SO₄ added (201.7*.48=96.8 g, 1.0 mol) was 2:1. Phases were separated. A viscous, oily water phase (turbid) was obtained. Seed, obtained as described in Example 1 was added to the aqueous phase. Massive crystallization started, in the course of less than one minute the mixture was a solid cake of white crystals. The wet cake of crystals was dried in vacuum at 20° C. The assay of D-phenylglycine methyl ester in the crystals was 73% (w/w), theoretical assay of D-phenylglycine methyl ester in the hemi sulfuric acid salt of D-phenylglycine methyl ester is 100*2*165.2/(2*165.2+98)=77%.

### Example 3

### Solubility of (PGMH)₂SO₄ in Water as a Function of Temperature

In the preparation of (PGMH)₂SO₄ as described in Example 2 separation of the organic phase at pH=4.2 is done while (PGMH)₂SO₄ is supersaturated. At some point in time, crystallization may start before the organic layer is separated from the aqueous phase. In order to design a process that will avoid crystallization of (PGMH)₂SO₄ in the presence of organic solvent, and controlled crystallization after separation of the organic phase, solubility of (PGMH)₂SO₄ as a function of temperature was investigated. The hemi sulfuric acid salt of D-phenylglycine methyl ester (1 g), obtained as described in Example 2 was mixed with water (2 g) at 20° C. and the solid material dissolved. Additional hemi sulfuric acid salt of D-phenylglycine methyl ester (1 g) was added and the mixture was agitated at 20° C. for 25 minutes. Not all solid was dissolved. An aliquot of approximately 0.5 mL of supernatant was filtered, and in the filtrate the concentration of hemi sulfuric acid salt of D-phenylglycine methyl ester was determined by HPLC. The rest of the mixture was stirred at 3° C. Water (2 mL) was added to allow mixing. Additional hemi sulfuric acid salt of D-phenylglycine methyl ester (0.5 g) was added and the mixture was agitated for 30 minutes. Not all solid was dissolved. An aliquot of approximately 0.5 mL of supernatant was filtered and in the filtrate the concentration of hemi sulfuric acid salt of D-phenylglycine methyl ester was determined by HPLC. The results of HPLC analysis are presented in Table 1.

TABLE 1
Solubility of hemi sulfuric acid salt of D-phenylglycine methyl ester in
water as a function of temperature

| | Hemi sulfuric |
|---|---|
| T (° C.) | acid salt of D-phenylglycine methyl ester (g)/kg of solution |
| 20 | 478 |
| 3 | 268 |

The solubility at 20° C. should allow phase separation after mixing D-phenylglycine methyl ester in organic solvent plus aqueous H₂SO₄ at pH=4.2 at 20° C. Subsequent cooling to 3° C. of the aqueous phase will result in crystallization of about 478-268=210 g of hemi sulfuric acid salt of D-phenylglycine methyl ester per kg of mixture. After isolation of hemi sulfuric acid salt of D-phenylglycine methyl ester from the crystal suspension at 3° C., the mother liquor can be re-used for extraction of D-phenylglycine methyl ester in organic solvent with water/H₂SO₄/mother liquor.

### Example 4

### Preparation of Cephalexin Using (PGMH)₂SO₄ vs PGM in Solution

7-Aminodeacetoxycephalosporanic acid (7-ADCA, 55.4 g) was suspended in water (237 mL) and the temperature was controlled at 20° C. The mixture was stirred for 5 min while maintaining the pH at 7.0 by the addition of an aqueous solution of ammonia (25%). Immobilized enzyme (comprising mutant 1 as described in U.S. Pat. No. 8,541,199; 18.7 g) was added together with water (25 mL). Next, solid (PGMH)₂SO₄ (61.5 g) was dosed at a constant rate in 200 min. whilst the pH was maintained at 7.0 by the addition of an aqueous solution of ammonia (25%) or with an aqueous solution of sulfuric acid (30%) once all (PGMH)₂SO₄ was added. After 230 min., the pH was adjusted to 5.8 by addition of an aqueous solution of sulfuric acid (30%). During the course of the reaction samples were taken and analyzed by HPLC with the results as outlined in Table 2.

TABLE 2
Formation of cephalexin from 7-ADCA using solid (PGMH)₂SO₄

| | | | | Cepha- | Conver- | | |
|---|---|---|---|---|---|---|---|
| Time | PG | 7-ADCA | PGM | lexin | sion | | |
| (min) | (%) | (%) | (%) | (%) | (%) | Ratio | S/H |
| 120 | 0.34 | 3.5 | 0.62 | 12.74 | 69.2 | 0.805 | 16.3 |
| 150 | 0.43 | 2.65 | 0.75 | 15.63 | 78.4 | 0.913 | 15.8 |
| 180 | 0.44 | 1.83 | 0.63 | 17.87 | 85.8 | 0.970 | 17.7 |
| 201 | 0.53 | 0.67 | 0.28 | 19.68 | 94.8 | 1.035 | 16.2 |
| 230 | 0.58 | 0.5 | 0 | 20.03 | 96.1 | 1.025 | 15.0 |
| 235 | 0.59 | 0.45 | 0 | 20.42 | 96.6 | 1.030 | 15.1 |

Components are given in weight %
Conversion: 100*moles cephalexin/(moles cephalexin + 7-ADCA)
Ratio: (moles cephalexin + PGM + PG)/(moles cephalexin + 7-ADCA)
S/H: Synthesis/Hydrolysis ratio, or moles cephalexin/moles PG

For comparative reasons the above cephalexin protocol was repeated however using PGM solution (as obtained in by Example 8 of U.S. Pat. No. 8,541,199; 100.7 g; assay PGM: 44%) instead of solid (PGMH)₂SO₄. In addition the initial suspension of 7-ADCA was in 187 mL of water instead of 237 mL During the course of the reaction samples were taken and analyzed by HPLC with the results as outlined in Table 3.

TABLE 3
Formation of cephalexin from 7-ADCA using PGM in solution

| | | | | Cepha- | Conver- | | |
|---|---|---|---|---|---|---|---|
| Time | PG | 7-ADCA | PGM | lexin | sion | | |
| (min) | (%) | (%) | (%) | (%) | (%) | Ratio | S/H |
| 120 | 0.49 | 2.72 | 0.41 | 14.13 | 76.2 | 0.869 | 12.6 |
| 150 | 0.55 | 2.54 | 0.23 | 15.54 | 79.1 | 0.879 | 12.3 |
| 180 | 0.64 | 2.08 | 0.46 | 17.52 | 83.9 | 0.955 | 11.9 |
| 205 | 0.72 | 1.36 | 0.47 | 18.26 | 89.2 | 1.021 | 11.0 |
| 230 | 0.77 | 0.86 | 0.07 | 19.08 | 93.2 | 1.026 | 10.8 |
| 235 | 0.79 | 0.75 | 0.02 | 19.67 | 94.2 | 1.031 | 10.8 |

Legend: As in Table 2

Inspection of Tables 2 and 3 revealed that the use of solid (PGMH)₂SO₄ resulted in significantly better results over the use of PGM in solution, in terms of maximum cephalexin formation, maximum conversion and overall S/H ratio.

1. The hemi sulfuric acid salt of D-phenylglycine methyl ester.

2. The hemi sulfuric acid salt according to claim 1 having an XRD powder diffraction pattern comprising peaks at 6.1±0.2 degrees 2-theta, 12.1±0.2 degrees 2-theta, 18.8±0.2 degrees 2-theta and 24.1±0.2 degrees 2-theta.

3. The hemi sulfuric acid salt according to claim 2 further comprising peaks at 7.9±0.2 degrees 2-theta, 14.4±0.2 degrees 2-theta, 15.6±0.2 degrees 2-theta, 16.7±0.2 degrees 2-theta, 19.5±0.2 degrees 2-theta and 25.6±0.2 degrees 2-theta.

4. A method for the preparation of the hemi sulfuric acid salt of D-phenylglycine methyl ester comprising the steps of:
(a) contacting a solution of D-phenylglycine methyl ester in an organic solvent with sulfuric acid;
(b) isolating the hemi sulfuric acid salt of D-phenylglycine methyl ester from the mixture obtained in step (a),
wherein the molar amount of sulfuric acid in step (a) is from 0.4 to 0.6 relative to the molar amount of D-phenylglycine methyl ester.

5. The method according to claim 4 wherein step (a) is followed by separation of the aqueous phase and step (b) is carried out on said aqueous phase.

6. The method according to claim 5 wherein said aqueous phase obtained after step (a) is subjected to crystallization.

7. The method according to claim 6 wherein said crystallization is carried out by lowering the temperature of said aqueous phase obtained after step (a).

8. The method according to claim 6 wherein said crystallization is carried out at a temperature of from −5 to 15° C.

9. The method according to claim 5 wherein the aqueous phase remaining after said isolating in step (b) is added to the mixture of step (a).

10. The method according to claim 4 wherein the solubility in water of said organic solvent is from 0% (w/w) to 25% (w/w) and the polarity index of said organic solvent is from 1 to 5.

11. The method according to claim 10 wherein said polarity index is from 2 to 3.

12. The method according to claim 10 wherein said solvent is chosen from the group consisting of butyl acetate, diethyl ether, ethyl acetate, methyl isobutyl ketone, methyl tert-butyl ether and mixtures thereof.

13. A method for preparing one or more of ampicillin, cefaclor or cephalexin comprising contacting the hemi sulfuric acid salt of D-phenylglycine methyl ester with 6-aminopenicillanic acid, 7-amino-3-chloro-3-cephem-4-carboxylate or 7-aminodeacetoxycephalosporanic acid, respectively in the presence of a penicillin acylase.

14. (canceled)
